(19) United States
(12) Patent Application Publication
(10) Pub. No.: US 2020/0199600 A1
(43) Pub. Date: Jun. 25, 2020

(54) ORTHOGONAL GAMMA PNA DIMERIZATION DOMAINS EMPOWERING DNA BINDERS WITH COOPERATIVITY AND VERSATILITY MIMICKING THAT OF THE TRANSCRIPTION FACTOR PAIRS

(71) Applicants: Wei-Che HSIEH, Pittsburgh, PA (US); Danith H. LY, Pittsburgh, PA (US); Hiroshi SUGIYAMA, Kyoto (JP); Zutao YU, Kyoto (JP)

(72) Inventors: Wei-Che HSIEH, Pittsburgh, PA (US); Danith H. LY, Pittsburgh, PA (US); Hiroshi SUGIYAMA, Kyoto-shi (JP); Zutao YU, Kyoto-shi (JP)

(21) Appl. No.: 16/676,613

(22) Filed: Nov. 7, 2019

Related U.S. Application Data
(60) Provisional application No. 62/766,873, filed on Nov. 7, 2018.

(51) Int. Cl. C12N 15/67; C12N 5/00; C07D 403/14

(52) U.S. Cl. CPC C12N 15/67; C07D 403/14; C12N 5/0006

(57) ABSTRACT

A pair of pyrrole-imidazole polyamides conjugated with nucleic acid-based cooperation system is provided.

### TECHNICAL FIELD

The present disclosure relates to a pyrrole-imidazole polyamide conjugated with nucleic acid-based cooperation system. Specifically, the disclosure is directed to a powerful cooperative DNA binding system Pip-NaCo (pyrrole-imidazole polyamides conjugated with nucleic acid-based cooperation system). The system showed that the cooperativity is highly comparable with natural system.

### BACKGROUND OF THE INVENTION

Spatial-temporal gene expression are precisely controlled by more than 1000 transcription factors (TFs) that recognize around 200 short DNA motifs in mammals. See A. Jolma, J. Yan, T. Whitington, J. Toivonen, Kazuhiro R. Nitta, P. Rastas, E. Morgunova, M. Enge, M. Taipale, G. Wei, K. Palin, Juan M. Vaquerizas, R. Vincentelli, Nicholas M. Luscombe, Timothy R. Hughes, P. Lemaire, E. Ukkonen, T. Kivioja, J. Taipale, Cell 2013, 152, 327-339. Usually, TFs function as cooperative TF-TF pairs through formation of noncovalently bound homo-/heterodimers, which occur in different orientations and/or gap spacings relative to each other. See E. Morgunova, J. Taipale, Curr. Opin. Struct. Biol. 2017, 47, 1-8; and G. Stampfel, T. Kazmar, O. Frank, S. Wienerroither, F. Reiter, A. Stark, Nature 2015, 528, 147-151. The effect of versatile gap spacings between TF-TF pairs on gene activation have been well characterized, (C. K. Ng, N. X. Li, S. Chee, S. Prabhakar, P. R. Kolatkar, R. Jauch, Nucleic Acids Res. 2012, 40, 4933-4941.) and TF pairs flexibly facilitate mutual binding in diverse binding orientations. See A. Jolma, Y. Yin, K. R. Nitta, K. Dave, A. Popov, M. Taipale, M. Enge, T. Kivioja, E. Morgunova, J. Taipale, Nature 2015, 527, 384-388.) For example, the binding site of the C-clamp of T-cell factor (TCF), which is indispensable for specific gene activation via the Wnt pathway, can act as a helper by swinging to localize upstream or downstream of the classical high-mobility group (HMG) binding sites. See N. P. Hoverter, M. D. Zeller, M. M. McQuade, A. Garibaldi, A. Busch, E. M. Selwan, K. J. Hertel, P. Baldi, M. L. Waterman, Nucleic Acids Res. 2014, 42, 13615-13632; and A. J. Ravindranath, K. M. Cadigan, Cancers 2016, 8, 74. Programmable molecules, e.g., nucleic acid analogues, pyrrole-imidazole polyamides (PIPs), short peptides, and peptide—small molecule covalent conjugates, have been widely applied to disrupt individual TF-DNA interactions. See J. M. Gottesfeld, L. Neely, J. W. Trauger, E. E. Baird, P. B. Dervan, Nature 1997, 387, 202-205; A. Dragulescu-Andrasi, S. Rapireddy, G. He, B. Bhattacharya, J. J. Hyldig-Nielsen, G. Zon, D. H. Ly, J. Am. Chem. Soc. 2006, 128, 16104-16112; J. Taniguchi, G. N. Pandian, T. Hidaka, K. Hashiya, T. Bando, K. K. Kim, H. Sugiyama, Nucleic Acids Res. 2017, 45, 9219-9228; E. Pazos, J. Mosquera, M. E. V#zquez, J. L. MascareÇas, Chem Bio Chem 2011, 12, 1958-1973; M. Wang, Y. Yu, C. Liang, A. Lu, G. Zhang, Int. J. Mol. Sci. 2016, 17, 779; and O. Vàzquez, M. E. Vàzquez, J. B. Blanco, L. Castedo, J. L. MascareÇas, Angew. Chem. Int. Ed. 2007, 46, 6886-6890; Angew. Chem. 2007, 119, 7010-7014. However, they cannot block interactions between collaborative TF pairs and DNA. Therefore, new strategies, especially the incorporation of modules allowing cooperative interactions between DNA binders, are needed to address these challenges in a deliberate and precise manipulation of gene expression patterns. See M. Ueno, A. Murakami, K. Makino, T. Morii, J. Am. Chem. Soc. 1993, 115, 12575-12576; M. D. Distefano, P. B. Dervan, Proc. Natl. Acad. Sci. USA 1993, 90, 1179-1183; J. B. Blanco, V. I. Dodero, M. E. Vàzquez, M. Mosquera, L. Castedo, J. L. Mascare Ças, Angew. Chem. Int. Ed. 2006, 45, 8210-8214; Angew. Chem. 2006, 118, 8390-8394; M. I. Sànchez, J. Mosquera, M. E. Vàzquez, J. L. MascareÇas, Angew. Chem. Int. Ed. 2014, 53, 9917-9921; Angew. Chem. 2014, 126, 10075-10079; and D. Chang, K. T. Kim, E. Lindberg, N. Winssinger, Bioconjugate Chem. 2018, 29, 158-163.

PIP is currently the best characterized programmable DNA minor-groove binder, and it binds according to the rules of Py/Im with C/G, Im/Py with G/C, and Py/Py with A/T and T/A′ See J. W. Trauger, E. E. Baird, P. B. Dervan, Nature 1996, 382, 559-561. Recently, we reported a PIP conjugating host-guest cooperation based system, named Pip-HoGu, for targeting cooperative TF pairs (FIG. 1). See Z. Yu, C. Guo, Y. Wei, K. Hashiya, T. Bando, H. Sugiyama, J. Am. Chem. Soc. 2018, 140, 2426-2429. From in vitro and cell-based assays, Pip-HoGu exhibits potent cooperation with spacings of <5 nt between two DNA binders. The essence of cooperativity in DNA binding is that the addition of the partner strand can highly stabilize binding of the overall complexes, and the difference in ability to form complexes in the absence or presence of the partner strand reflects the magnitude of cooperativity. See S. F. Singleton, P. B. Dervan, Biochemistry 1992, 31, 10995-11003. In addition, the dual binders should prefer to bind the DNA sites containing dual target sites simultaneously in a proper binding orientation, while decreasing the ratio of monomer binding. Moreover, cooperativity should be capable of avoiding mismatch binding to an extent, and it should also bind degenerate DNA sites with reasonable affinity under some conditions. See A. Jolma, Y. Yin, K. R. Nitta, K. Dave, A. Popov, M. Taipale, M. Enge, T. Kivioj a, E. Morgunova, J. Taipale, Nature 2015, 527, 384-388. There are several potential limitations of the previously reported Pip-HoGu system. For example, it is not practical for the case of spacings >5 nt and, more significantly, alternative orientations. The cooperation binding energy of the host-guest system could not be finely tuned independently Id. Moreover, the interaction of host-guest moieties is electrostatic and hydrophobic interactions, rather than residue-specific interactions.

### SUMMARY OF THE INVENTION

Here, we expanded the cooperation module from host-guest system to oligonucleotide directed sequence specific recognition moiety. See M. D. Distefano, P. B. Dervan, Proc. Natl. Acad. Sci. USA 1993, 90, 1179-1183. Peptide nucleic acid (PNA) is an enzymatically stable, tight-binding, synthetically versatile, and informationally interfaced nucleic acid platform. See M. Egholm, O. Buchardt, L. Christensen, C. Behrens, S. M. Freier, D. A. Driver, R. H. Berg, S. K. Kim, B. Norden, P. E. Nielsen, Nature 1993, 365, 566-568; 0. Berger, E. Gazit, Pept. Sci. 2017, 108, e22930; and S. Ellipilli, K. N. Ganesh, J. Org. Chem. 2015, 80, 9185-9191, Several groups have made significant headway using gamma-backbone PNA modifications, which transform a randomly folded PNA into a preorganized right-handed (RH) or left-handed (LH) helix. See B. Sahu, V. Chenna, K. L. Lathrop, S. M. Thomas, G. Zon, K. J. Livak, D. H. Ly, J. Org. Chem. 2009, 74, 1509-1516; A. Dragulescu-Andrasi, S. Rapireddy, B. M. Frezza, C. Gayathri, R. R. Gil, D. H. Ly, J. Am. Chem. Soc. 2006, 128, 10258-10267; D. R. Jain, L. Anandi V, M. Lahiri, K. N. Ganesh, J. Org. Chem. 2014, 79, 9567-9577; A. Manna, S. Rapireddy, G. Sureshkumar, D. H. Ly, Tetrahedron 2015, 71, 3507-3514; E. A. Englund, D. H. Appella, Angew. Chem. Int. Ed. 2007, 46, 1414-1418; Angew. Chem. 2007, 119, 1436-1440; and S. Sforza, T. Tedeschi, R. Corradini, R. Marchelli, Eur. J. Org. Chem. 2007, 5879-5885.

More intriguingly, LH γPNA can hybridize to partner strands containing a complementary sequence and matching helical sense; however, they do not cross-hybridize with RH γPNA, DNA, or RNA. See I. Sacui, W.-C. Hsieh, A. Manna, B. Sahu, D. H. Ly, J. Am. Chem. Soc. 2015, 137, 8603-8610. Such orthogonal properties and programmability endow LH γPNA with the desired cooperative modules to mimic TF-pair cooperation for molecular assembly and computing while avoiding cross-hybridization with the host's endogenous genetic materials.

In this context, we envisaged the integration of programmable PIPs with an orthogonal LH γPNA cooperative system, named Pip-NaCo, to mimic the natural versatile binding systems of TF pairs (FIG. 1). Distinct from Pip-HoGu, Pip-NaCo cooperation is a specific interaction of hydrogen bond with base pairing, which could theoretically cover a spacing as long as its linker length. Results show a minimum cooperation of −3.27 kcal mol⁻¹, and can flexibly change PIPs-binding orientation and conjugation sites. Furthermore, the tunability of PNA length, orthogonality, and toehold strand displacement performance further make Pip-NaCo a fascinating tool for mimicking cooperation of transcription factor pairs.

Synthetic molecules capable of DNA binding and mimicking cooperation of transcription factor (TF) pairs have long been considered a promising tool for manipulating gene expression. Our previously reported Pip-HoGu system, a programmable DNA binder pyrrole-imidazole polyamides (PIPs) conjugated to host-guest moiety, defined a general framework for mimicking cooperative TF pair-DNA interactions. Here, we supplanted the cooperation modules with left-handed (LH) γPNA modules: i.e., PIPs conjugated with nucleic acid-based cooperation system (Pip-NaCo). LH γPNA was chosen because of its bioorthogonality, sequence-specific interaction, and high binding affinity toward the partner strand. From the results of the Pip-NaCo system, cooperativity is highly comparable to the natural TF pair-DNA system, with a minimum energetics of cooperation of −3.27 kcal mol⁻¹. Moreover, through changing the linker conjugation site, binding mode, and the length of γPNAs sequence, the cooperative energetics of Pip-NaCo can be tuned independently and rationally. The current Pip-NaCo platform might also have the potential for precise manipulation of biological processes through the construction of triple to multiple heterobinding systems.

### BRIEF DESCRIPTION OF THE DRAWINGS

FIG. 1 is a schematic illustration of the current research design. Based on our previously reported Pip-HoGu system, the host-guest interaction domain was replaced with a nucleic acid-based sequence-specific interaction domain, termed Pip-NaCo.

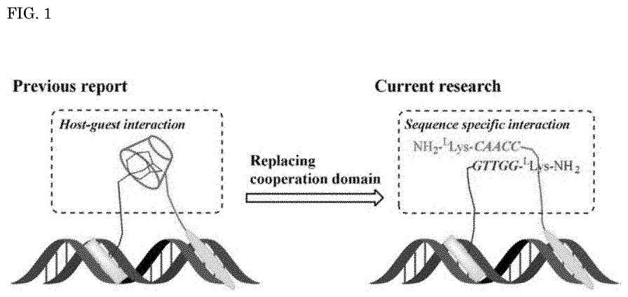

FIG. 2 is a Schematic representation of cooperative interactions of two components of the Pip-NaCo assembly (PP1 and PP2) with dsDNA backbone. Thick solid lines represent the dsDNA backbone of the target site and associated oligonucleotides. The thin module array represents oligonucleotide sequence specific hydrogen bonds. n=gap distance. The dimerization domain of LH^{1-MP}γPNA consisting of 5 nt sequence is shown as colored, bold, and italic letters. (Bottom) Chemical structures of PP1 and PP2.

FIG. 3 is a CD spectra of nonhybridized PP1 and PP2, each at 10.0 μM concentration, and the corresponding PP1-PP2 at 5.0 μM concentration of each strand, recorded at 22° C. The CD spectrum was recorded from 230-300 nm. CD measurements were prepared in sodium phosphate buffer (10 mM sodium phosphate, 0.1 mM EDTA, 100 mM NaCl, pH 7.2).

FIG. 4 shows the spacing-dependent manner of cooperative binding of Pip-NaCo. A) The DNA oligomers (ODNs) used in the Tₘ assay, including positive (Mode A, ODN1′P-ODN8P) and negative (Mode B, ODN1′N-ODN8N) binding sequences. The gap distance (green) is the number of base pairs between the binding sites of PP1 (blue) and PP2 (red). Spacing is the distance between two PNA conjugation sites: i.e., spacing equals the gap distance in Mode A, but in Mode B, it equals the gap distance plus two PIP-binding sites. The upper chart shows only the forward DNA strand and omits the complementary DNA strand. B,C) The gel-shift behavior of all the positive binding sequences in Mode A (B) and negative-binding sequences in Mode B (C) with PP1-PP2. ODN concentration: 1.0 μM. Compound concentration: 10.0 μM. Black arrow: ODN2P; red: ODN2P/PP1-PP2. Except special illustration, the gel bands were stained with SYBR gold and quantified with a FujiFilm FLA-3000G fluorescent imaging analyzer. Unless otherwise stated, all samples used in the electrophoretic mobility shift assay measurements were prepared in sodium phosphate buffer (10 mM sodium phosphate, 0.1 mM EDTA, 100 mM NaCl, pH 7.2).

In the Positive binding mode column, the sequences shown from top to bottom are SEQ ID NOs:5-13, respectively. In the Negative binding mode column, the sequences shown from top to bottom are SEQ ID NOs:14-22, respectively.

FIG. 5 shows EMSA results illustrating the cooperation of Pip-NaCo in different binding modes. A) Schematic illustration of PP1-PP2 binding with ODNs in Mode C and D. Mode C shows SEQ ID NO:1 and its reverse strand. Mode D shows SEQ ID NO:2 and its reverse strand. B) The gel-shift behavior of PP1-PP2 with ODNs of Mode A-2P (lanes 1-4), Mode A-6P (lanes 5-7), Mode C (lanes 8-10), and Mode D (lanes 11-13). The gap distance (green) is the number of base pairs between the binding sites of PP1 (blue) and PP2 (red). Spacing is the distance between two PNA conjugation sites, i.e., in Mode C, it equals the gap distance plus PP2 binding sites. ODN concentrations: 1.0 μM. Compound concentration: 10.0 μM.

FIG. 6 shows Quantitative EMSAs evaluating the cooperation of Pip-NaCo. A) Quantitative EMSA of Mode C with PP1 at various concentrations (top) and PP1 supplemented with 5.0 μm PP2 (bottom). ODN concentration: 100 nM. Compound concentrations range from 0.1 to 10.0 μM (10-fold concentrations from 100 nM are showed in the Figure). FAM labeled forward strand (5′-FAM-AACTAGCCTAATGACGTATAT-3′) (SEQ ID NO:1) used for quantitative assay without SYBR gold staining. B) Binding isotherms obtained for PP1 alone (▪) and in the presence of PP2 (●) using quantitative EMSA. The data points were calculated from the average shift-band intensities of triplicate experiments. C) Equilibrium association constants and free energies for Mode C with PP1-PP2.

FIG. 7 shows the effect of PNA length on the cooperation of Pip-NaCo. A) Schematic illustration of Pip-NaCo assembly containing 7 nt γPNA sequences in Mode C. Mode C shows SEQ ID NO:1 and its reverse strand. Dashed square frame highlights the inserted nt. B) The gel-shift behavior of PP1-PP2 (lanes 1, 2), PP1-PP5 (lanes 3, 4), PP2-PP4 (lanes 5, 6), and PP4-PP5 (lanes 7, 8), with Mode C. ODN concentration: 1.0 μM. Compound concentration: 3.0 μM and 10.0 μM.

FIG. 8 shows toehold-mediated strand displacement assay of Pip-NaCo. A) Schematic illustration of toehold-mediated strand displacement assay with Pip-NaCo assemblies. gPNA5 is the competitive strand to displace PP2 binding. B) Toehold-mediated strand displacement assay in EMSA with Mode C. Mode C shows SEQ ID NO:1 and its reverse strand. ODN concentration: 1.0 μM. Compound concentrations are shown.

FIG. 9 shows the comparison of CD spectra between LH γPNA modified with γ-R-Me and PIP-PNA modified with γ-L-MP. This figure shows that PP2 (PIP2-^{R-Me}γPNA2, contains two thymines) is less stable than ^{R-Me}γPNA1, because thymine has the lowest base-stacking energy among the four nucleobases (J. Am. Chem. Soc. 2015, 137, 8603-8610). In another report (J. Org. Chem. 2011, 76, 5614-5627.), diethylene glycol (MP) substituent at γ-site enhance PNA pre-organization. Based on these results, we preclude that the higher stabilized LH conformation of PP2 (PIP2-^{S-MP}γPNA) is attributable to S-MP substituent at γ-site, by comparison with ^{R-Me}γPNA2 (R-Me substituent). Moreover, we agree that PIP conjugation will promotes the changes of CD pattern. But such changes might be destabilize the LH confirmation, rather than stabilize.

FIG. 10 shows the results of the gel shift assay of PIP1 and PP1, together with their respective chemical structures.

FIG. 11 shows the results of the gel shift assay of mismatch sequence with PP1-PP2. ODN-C is SEQ ID NO:2. ODN-CM is SEQ ID NO:3.

FIG. 12 shows the results of the binding affinity comparison between PP3-PP2 and PP1-PP1 with variable binding modes. Mode E shows SEQ ID NO:4 and its reverse strand. Mode F shows SEQ ID NO:1 and its reverse strand.

FIG. 13 shows the chemical structure of ^{L-MP}γPNA5.

FIG. 14 shows MASS data and HPLC data for Monomer A and Monomer T.

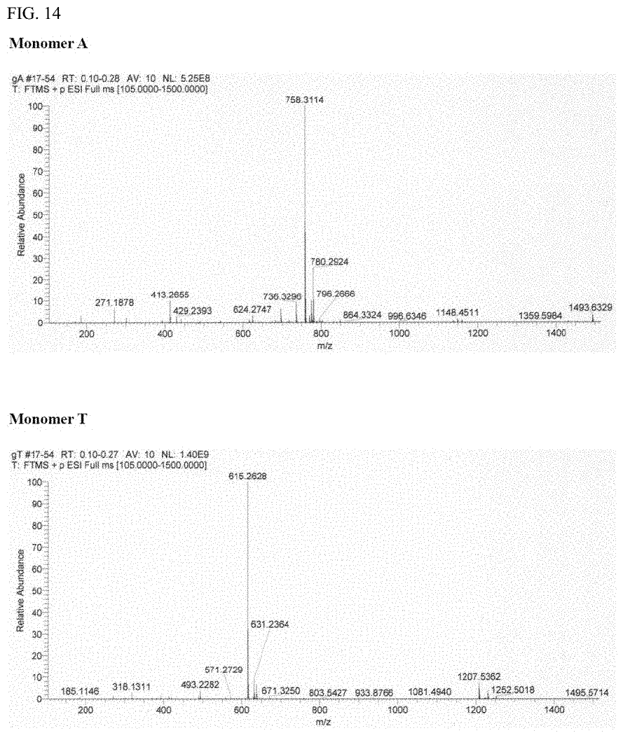

FIG. 15 shows MASS data and HPLC data for Monomer G and Monomer C.

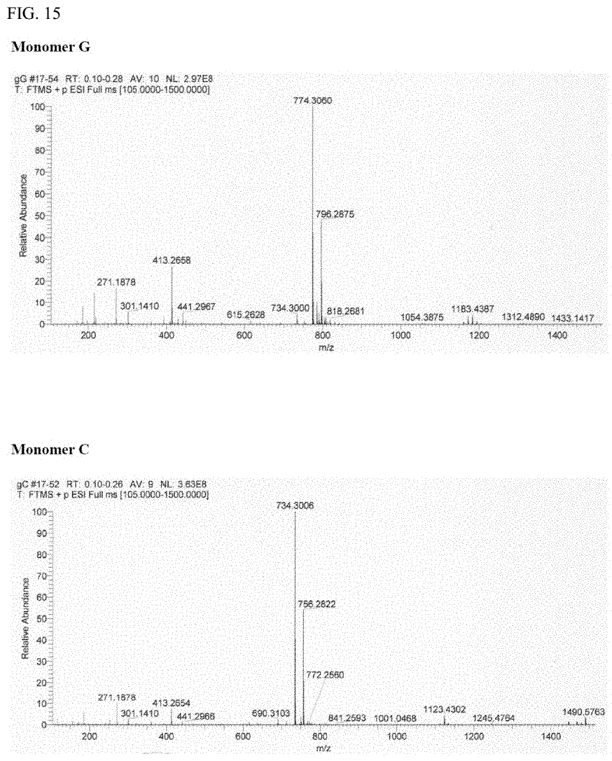

FIG. 16 shows PIP1 was obtained as a white powder. Overall yield is 4.5%. MALDI-TOF MS: m/z calcd for C₅₄H₆₁N₂₁NaO₁₂⁺[M+Na]⁺: 1219.2068; found: 1218.608. HPLC: t_{R}=16.675 min (0.1% TFA/MeCN, linear gradient 0-100%, 0-40 min).

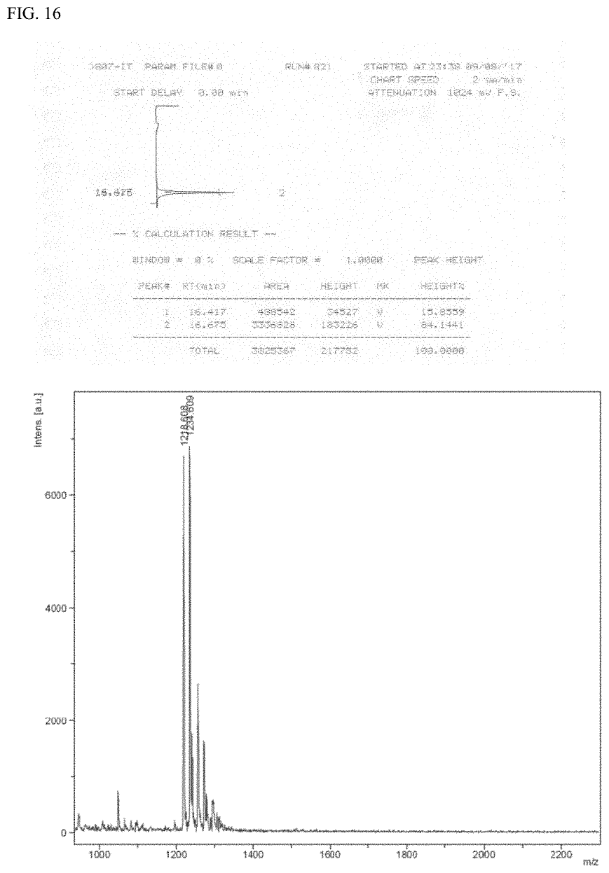

FIG. 17 shows PIP2 was obtained as a white powder. Overall yield is 13.5%. MALDI-TOF MS: m/z calcd for C₆₂H₇₈N₂₃O₁₃⁺[M+H]⁺: 1353.4540; found: 1351.968. HPLC: t_{R}=9.875 min (0.1% TFA/MeCN, linear gradient 0-100%, 0-20 min).

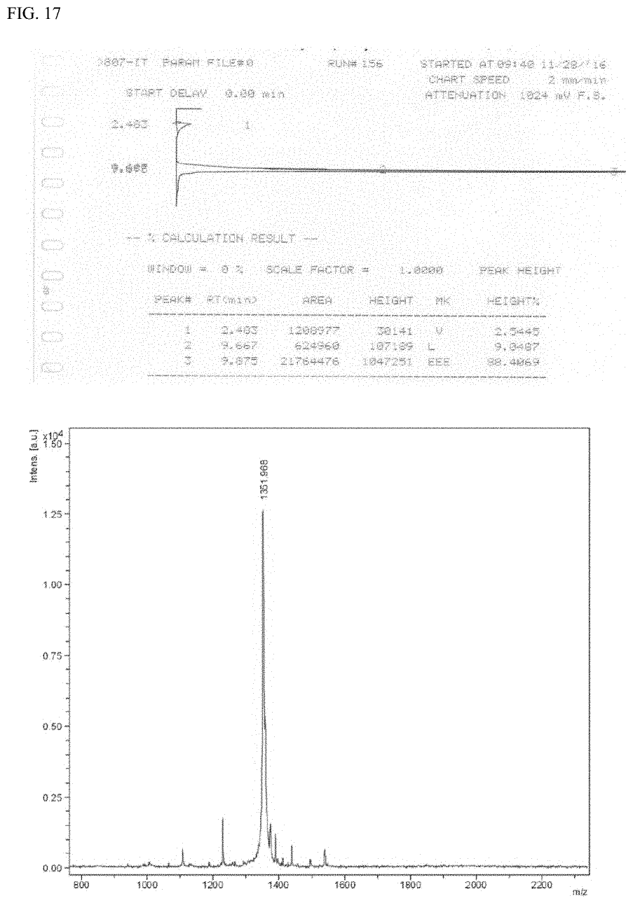

FIG. 18 shows PIP3 was obtained as a white powder. Overall yield is 5.5%. MALDI-TOF MS: m/z calcd for C₅₄H₆₂N₂₁O₁₂⁺[M+H]⁺: 1197.2250; found: 1196.898. HPLC: t_{R}=17.142 min (0.1% TFA/MeCN, linear gradient 0-100%, 0-40 min).

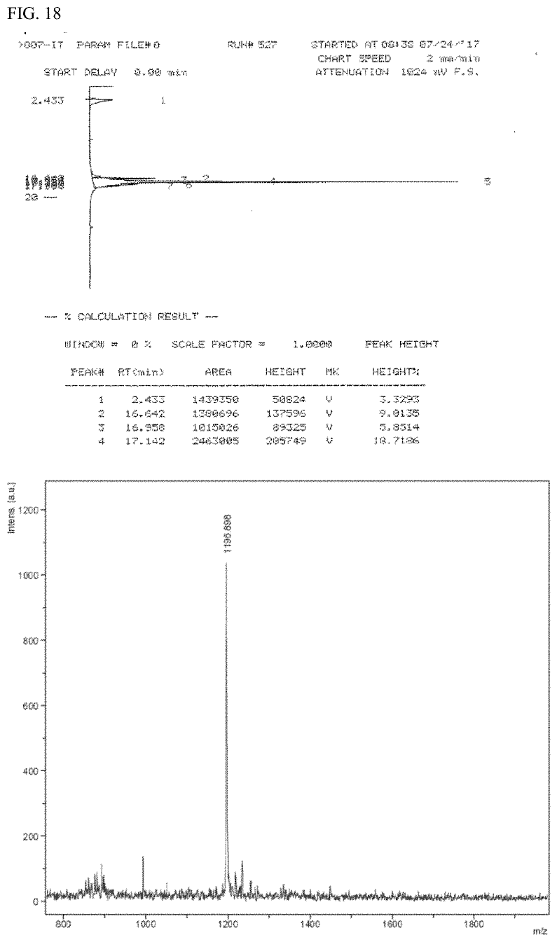

FIG. 19 shows PPI was obtained as a white powder. Yield is 35.1%. MALDI-TOF MS: m/z calcd for C₁₄₅H₂₀₅N₅₄O₄₄⁺[M+H]⁺: 3408.5690; found: 3405.703. HPLC: t_{R}=26.283 min (0.1% TFA/MeCN, linear gradient 0-50%, 0-40 min).

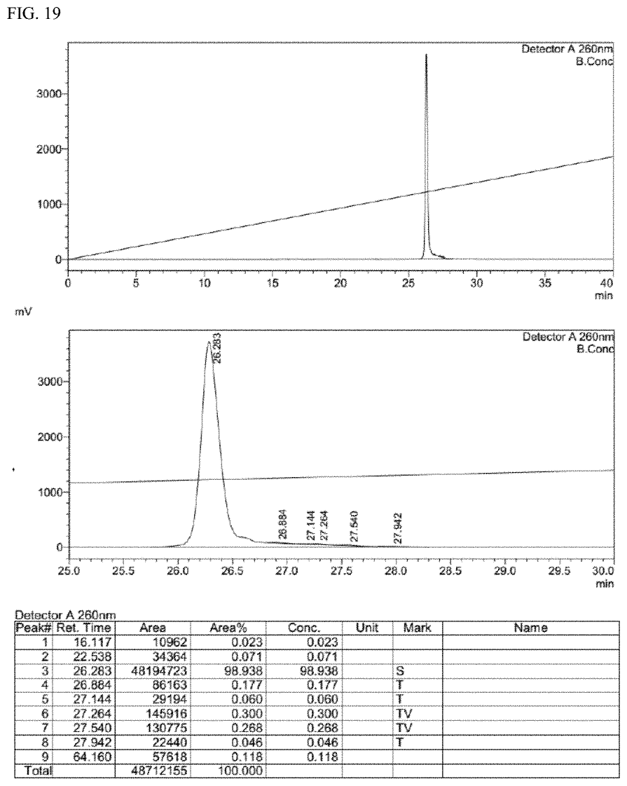

FIG. 20 is the mass spectra of PP1.

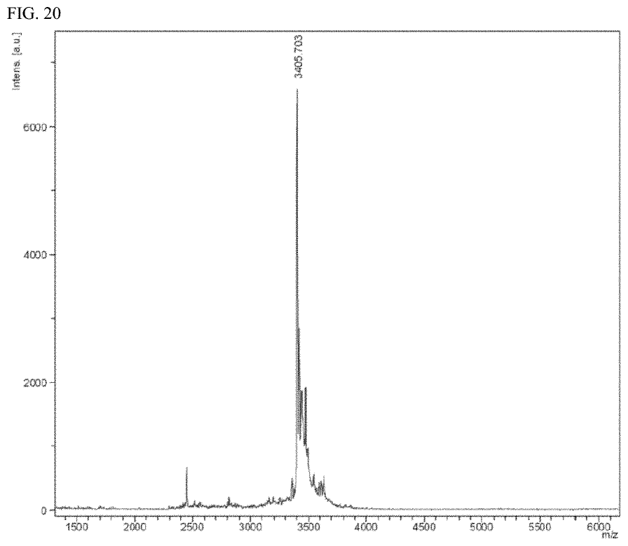

FIG. 21 shows PP2 was obtained as a white powder. Yield is 27.1%. MALDI-TOF MS: m/z calcd for C₁₅₆H₂₂₃N₅₆O₄₉⁺[M+H]⁺: 3666.8430; found: 3664.700. HPLC: t_{R}=26.990 min (0.1% TFA/MeCN, linear gradient 0-50%, 0-40 min).

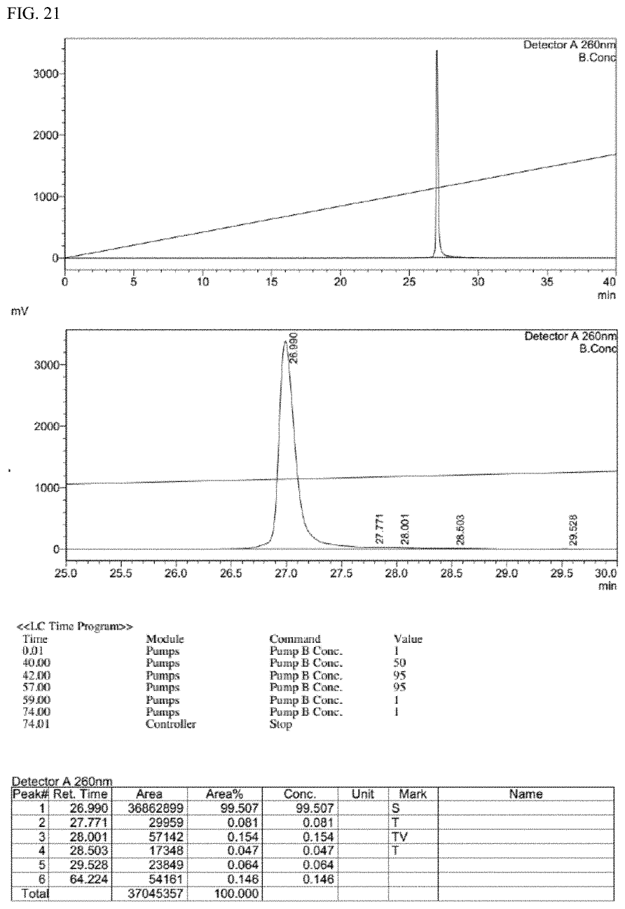

FIG. 22 is the mass spectra of PP2.

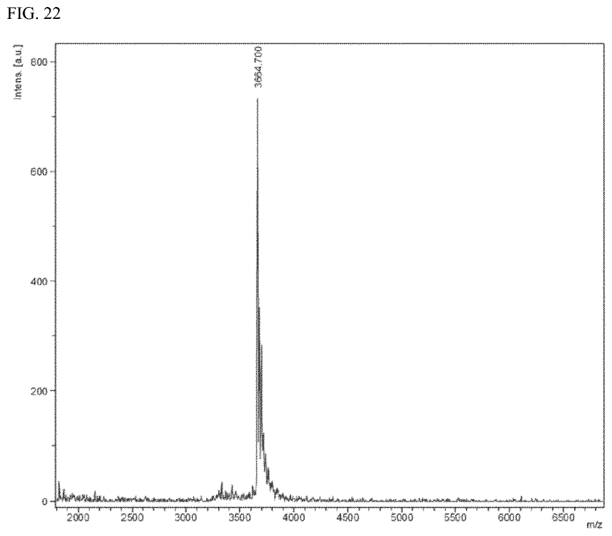

FIG. 23 shows PP3 was obtained as a white powder. Yield is 25.9%. MALDI-TOF MS: m/z calcd for C₁₄₈H₂₀₇N₅₄O₄₈⁺[M+H]⁺: 3510.6140; found: 3509.891. HPLC: t_{R}=27.200 min (0.1% TFA/MeCN, linear gradient 0-50%, 0-40 min).

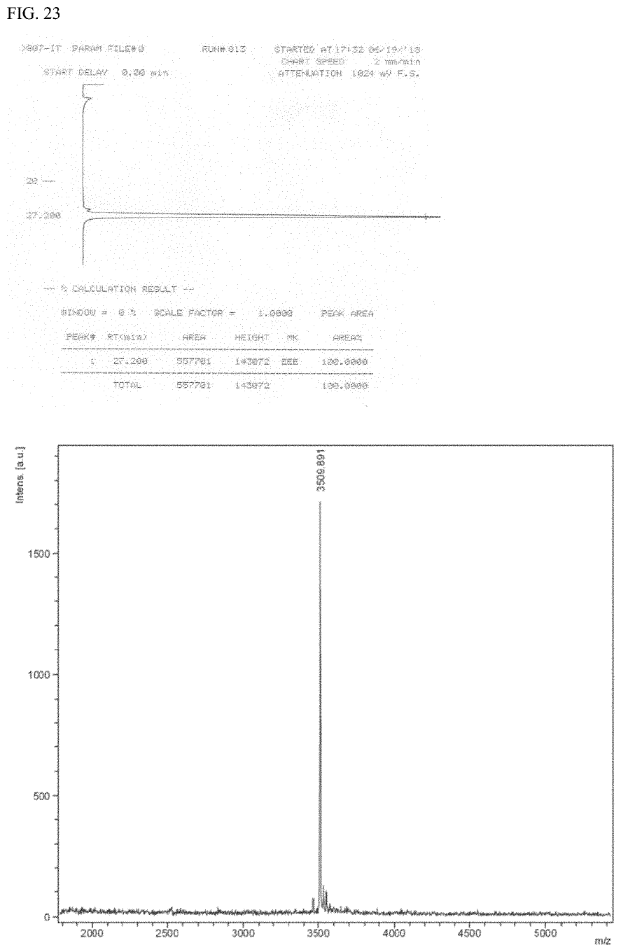

FIG. 24 shows PP4 was obtained as a white powder. Yield is 36.5%. MALDI-TOF MS: m/z calcd for C₁₇₇H₂₆₀N₆₈O₆₈⁺[M+H]⁺: 4227.3750; found: 4226.890. HPLC: t_{R}=26.083 min (0.1% TFA/MeCN, linear gradient 0-50%, 0-40 min).

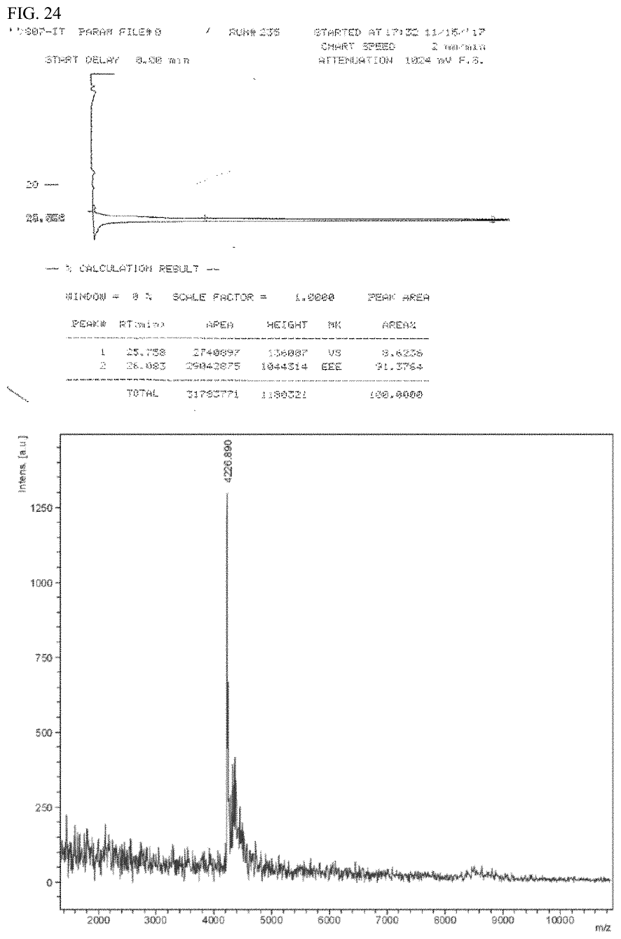

FIG. 25 shows PP5 was obtained as a white powder. Yield is 28.1%. MALDI-TOF MS: m/z calcd for C₁₈₆H₂₆₈N₆₆O₆₁⁺[M+H]⁺: 4405.59900; found: 4405.149. HPLC: t_{R}=26.242 min (0.1% TFA/MeCN, linear gradient 0-50%, 0-40 min).

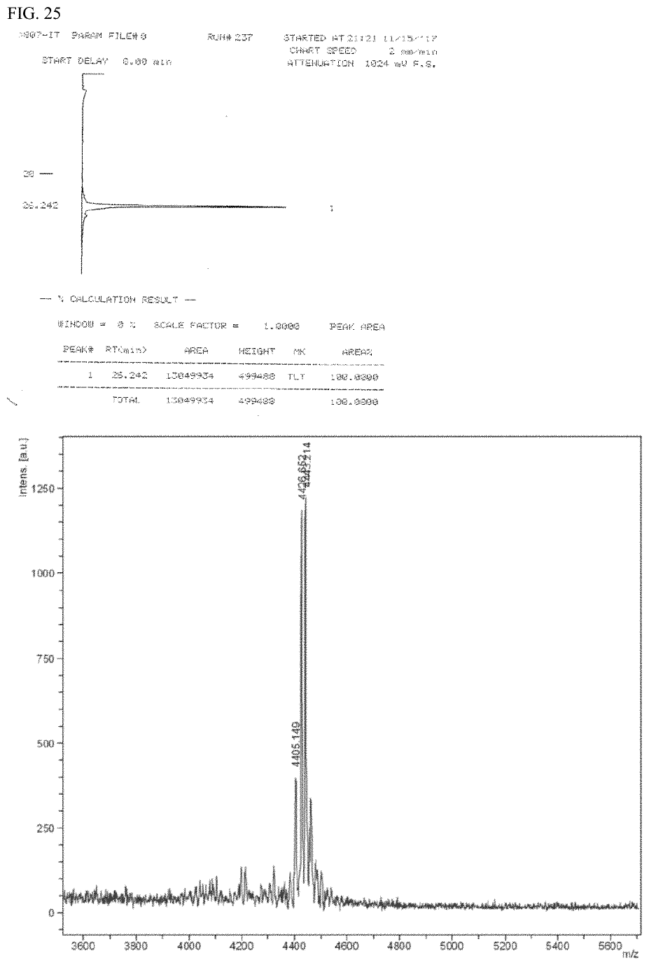

### DETAILED DESCRIPTION OF THE EMBODIMENTS

A conjugate of a pair of deoxyribonucleic acid (DNA) binders with a nucleic acid-based cooperation (NaCo) domain as disclosed herein may be a complex comprising a pair of DNA binders and a nucleic acid-based cooperation domain. The conjugate as disclosed herein is capable of binding to a DNA and mimicking cooperation of transcription factor (TF) pairs.

As used herein, DNA binders are a programmable DNA binder; and examples of DNA binders include but not limited to, nucleic acid analogues, pyrrole-imidazole polyamides (PIPs), short peptides, and peptide—small molecule covalent conjugates. Preferably, in an embodiment, its DNA binders are PIPs. The DNA binders included in a conjugate may be the same or different from each other. Each of DNA binders is designed to bind to a target site on a DNA. In general, the numbers and arrangement of pyrrole and imidazole moieties included in PIPs are arbitrary and can be appropriately determined by a person skilled in the art so that the PIPs can bind to target sites on a DNA.

As used herein, the term “nucleic acid-based cooperation system” represents a molecular assembly including a nucleic acid-based sequence-specific interaction domain that allows the components of the assembly to establish cooperative interactions thereof in their sequence specific manner. Examples of nucleic acid-based sequence-specific interaction domains include, but not limited to, e.g. “NaCo domain”. In an embodiment, the NaCo domain may have two left-handed (LH) gamma-PNA (γPNA) sequences which are complementary to each other. The γPNA sequences in the NaCo domain can have any length as long as they can establish cooperative interactions as intended. That is, the length of the sequences is adjustable, and it may be e.g. 3, 4, 5, 6, 7, 8, 9, or 10 nt length, or longer. Types and/or length of sequences of γPNA can be appropriately determined by a person skilled in the art, based on intended cooperation of the assembly, in light of the disclosure of the present specification.

In an embodiment, γPNAs may be modified at their γ-sites, with amino acids such as lysine, alanine, glutamic acid, diethylene glycol, or the like.

In an embodiment, the DNA binders included in the nucleic acid-based cooperation system are conjugated with NaCo domain via a linker. As used herein, the term “linker” refers to a moiety capable of serving as an attachment point for a chemical compound or moiety (i.e., a desired product) that is prepared by chemical synthesis, e.g. solid-phase synthesis. Examples of the linker include, but not limited to, e.g. PEG linker.

In an embodiment, two PIPs may serve as DNA binders; and linker conjugation sites of the PIPs may be positioned, for example, on their terminal or γ-turn. The linker conjugation site of one of the two PIPs may be on its terminal and the linker conjugation site of the other may be on its γ-turn, or the linker conjugation sites of both of the two PIPs may be on their terminals or γ-turns. In an embodiment, one of the two PIPs is connected to a linker on its terminal and the other is connected to the linker on its γ-turn.

In an embodiment, two PIPs bound to the target sites on a DNA may have various orientations to each other. For example, the two PIPs may be bound to a DNA in the same or opposite orientation, i.e., they run parallel to each other but with the same or opposite directionality.

In an embodiment, spacing between two γPNA conjugation sites on a target DNA may be adjustable.

According to the present disclosures, in an embodiment, the orthogonal gamma-PNA dimerization domains empower DNA binders with cooperativity and versatility mimicking that of transcription factor pairs.

### EXAMPLES

### Results and Discussion

### The Principle of the Pip-NaCo System

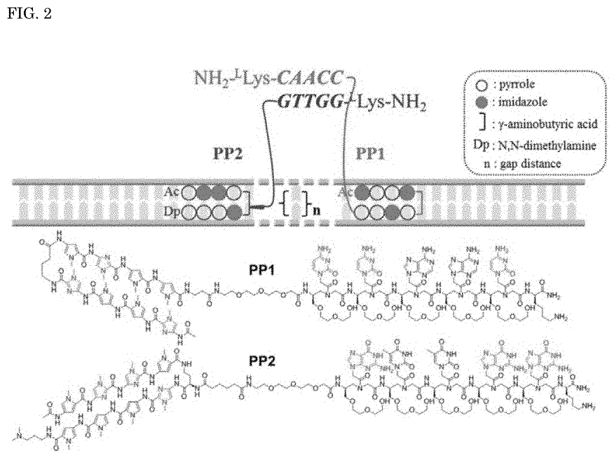

Two PIPs were designed to target their matching sequences (Z. Yu, C. Guo, Y. Wei, K. Hashiya, T. Bando, H. Sugiyama, J. Am. Chem. Soc. 2018, 140, 2426-2429.) and were individually conjugated with gamma-PNA domains (modified with L-diethylene glycol (L-MP) at the γ-site) through a PEG linker (FIG. 2). See. Kameshima, T. Ishizuka, M. Minoshima, M. Yamamoto, H. Sugiyama, Y. Xu, M. Komiyama, Angew. Chem. Int. Ed. 2013, 52, 13681-13684; Angew. Chem. 2013, 125, 13926-13929. The incorporation of a diethylene glycol unit was confirmed to enhance water solubility and reduce aggregation significantly. See B. Sahu, I. Sacui, S. Rapireddy, K. J. Zanotti, R. Bahal, B. A. Armitage, D. H. Ly, J. Org. Chem. 2011, 76, 5614-5627. The preorganized conformation of single-stranded γPNA and its binding with the respective matching strand could translate into higher affinity and sequence selectivity because of a reduction in the entropic penalty and an increase in backbone rigidity. See A. Dragulescu-Andrasi, S. Rapireddy, B. M. Frezza, C. Gayathri, R. R. Gil, D. H. Ly, J. Am. Chem. Soc. 2006, 128, 10258-10267. The full synthetic procedure and characterization of all conjugates of Pip-NaCo are provided in the Supporting Information. It is noteworthy that PIPs obtained from Fmoc solid-phase synthesis were incorporated onto γPNA tails on Boc solid-phase resin. See. Manna, S. Rapireddy, G. Sureshkumar, D. H. Ly, Tetrahedron 2015, 71, 3507-3514; and Z. Yu, J. Taniguchi, Y. Wei, G. N. Pandian, K. Hashiya, T. Bando, H. Sugiyama, Eur. J. Med. Chem. 2017, 138, 320-327.

The Pip-NaCo system was designed in a parallel binding orientation; that is, the γPNA duplex is parallel to dsDNA, and γPNA strands meeting each other in the manner of head-to-tail (FIG. 2). To our knowledge, Pip-NaCo sets the first example of the application of orthogonal, natural DNA-excluding LH γPNA conjugating with programmable DNA binders.

### Conformational Study

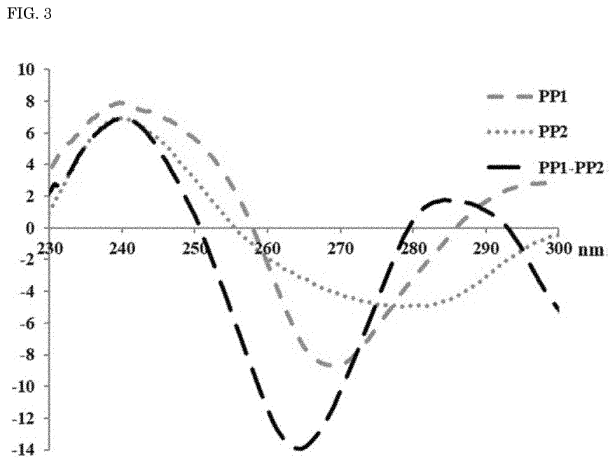

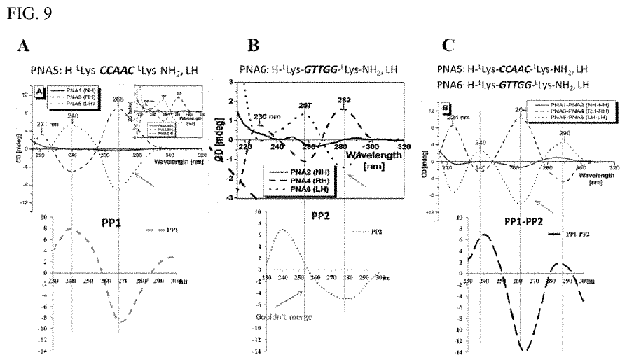

Circular dichroism (CD) experiments were conducted to determine the effect of PIP conjugation on the conformation of LH γPNA. See I. Sacui, W.-C. Hsieh, A. Manna, B. Sahu, D. H. Ly, J. Am. Chem. Soc. 2015, 137, 8603-8610. PP1 and PP2 modified with gamma-L-MP have the same nucleotide sequence as previously reported LH γPNA, which was modified with gamma-R-Me but without PIP conjugations. Id. By measuring the CD spectra and comparing them with those of LH γPNA modified with gamma-R-Me, we expected that the introduction of PIPs would not disturb the preorganized LH conformation of γPNA. As expected, PP1, PP2, and PP1-PP2 showed similar CD patterns; that is, a positive peak at around 240 nm and a negative peak at 265-275 nm, suggesting LH helical conformation (FIGS. 3, 9). Compared with the respective γPNA sequences (gamma-R-Me) without PIP conjugations, PP1 and PP1-PP2 exhibited highly identical CD profiles with unmodified single-strand γPNA sequences and their unmodified γPNA duplex sequences, respectively (FIG. 9 parts A and C). See U. Kadhane, A. I. S. Holm, S. V. Hoffmann, S. B. Nielsen, Phys. Rev. E 2008, 77, 021901. We conclude that PIP conjugations do not destroy the preorganized LH conformation of γPNA. Moreover, PP2 showed a canonical CD profile of LH conformation, but differed from its respective γPNA without PIP conjugation (FIG. 9, part B). Enhancement and stabilization of the preorganization of γPNA by substituting it with γ-L-MP might be one of the mechanisms. See B. Sahu, I. Sacui, S. Rapireddy, K. J. Zanotti, R. Bahal, B. A. Armitage, D. H. Ly, J. Org. Chem. 2011, 76, 5614-5627.

PP1 and PP2 showed moderate redshift of the CD signal in comparison to PP1-PP2 duplexes. The CD amplitudes of PP1-PP2 duplexes are higher than the sum of those for the two individual strands, and a third, a subtly positive peak emerges at 285 nm. Those results further support the notion that hybridization is likely to follow Fischer's “lock and key” hypothesis (E. Fischer, Ber. Dtsch. Chem. Ges. 1894, 27, 2985-2993.) and the formation of γPNA duplex facilitate and enhance the LH secondary conformations. See P. Wittung, M. Eriksson, R. Lyng, P. E. Nielsen, B. Norden, J. Am. Chem. Soc. 1995, 117, 10167-10173.

### Spacing-Dependent Manner of Cooperative Binding

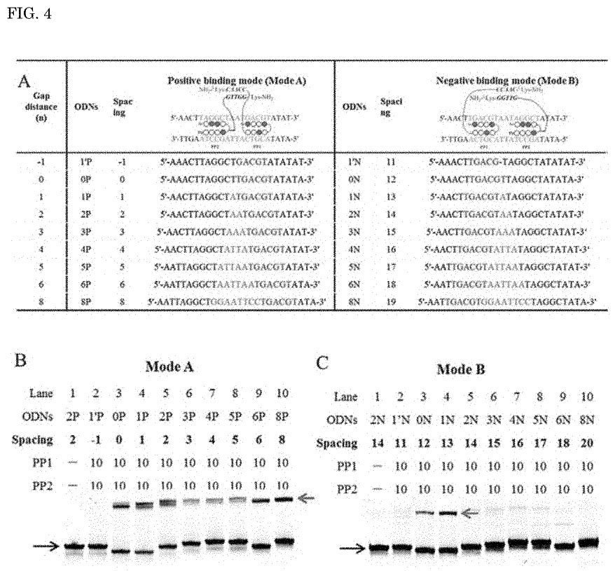

Pip-NaCo sequences were applied to the binding affinity assays with DNA sequences of ModeA and B (FIG. 4, part A). See Z. Yu, C. Guo, Y. Wei, K. Hashiya, T. Bando, H. Sugiyama, J. Am. Chem. Soc. 2018, 140, 2426-2429. The differences between Mode A and B originate from the relative positions of the PP1 and PP2 binding sites. More specifically, in Mode A, the γPNA conjugation sites are close to each other and can form duplexes after covering a short spacing (spacing=gap distance; FIG. 4, part B). However, in negative binding Mode B, the two γPNA domains have longer spacings that are equal to the gap distance plus two PIP-binding sites (spacing=gap distance+two PIP-binding sites; FIG. 4, part C, Table Si).

An electrophoretic mobility shift assay (EMSA) was conducted to determine the potency of the cooperative binding and how it was influenced by the spacings between the two PIP-binding sites, by direct visualization of the band-shift behavior upon formation of stable complexes. See R. Moretti, L. J. Donato, M. L. Brezinski, R. L. Stafford, H. Hoff, J. S. Thorson, P. B. Dervan, A. Z. Ansari, ACS Chem. Biol. 2008, 3,220-229. PP1-PP2 was equilibrated with DNA oligomers (ODNs) (Mode A and B) of varying spacings. Because of a PIP-binding steric conflict, no shifted band could be observed for ODNs with a 1 base pair deletion (ODN1′P and ODN1′N) (FIG. 4, parts B and C). However, the appearance of a shifted band showed that ODNs in Mode A (0-8 base pair gap distances) display potent cooperative binding. In striking contrast to the Pip-HoGu system (cooperation limited to spacing of 0-5 nt), significant band shifts were also observed for Mode B ODNs with spacing of 12 and 13 base pairs.

Furthermore, the EMSA data showed that, in Mode A, the shifted bands of the middle ODNs (ODN3P, ODN4P, and ODN5P) were weaker than those of the ODNs at both ends. These results can be explained when taken together with data from computational studies. Inserting a spacer between two PIP-binding sites will not only shift the linear distance but will also rotate them from the original position. In canonical BDNA, the addition of 1 nt rotates it 36 degrees alongside the DNA helix and it will have the same orientation again after the insertion of 10 nt. Based on computational studies, PP1 and PP2 are at the greatest angle distance in ODN4P, and further increases in spacings lead to the realignment of two PIPs, which is consistent with the observed results.

### Orientation Variation of Binding Sites

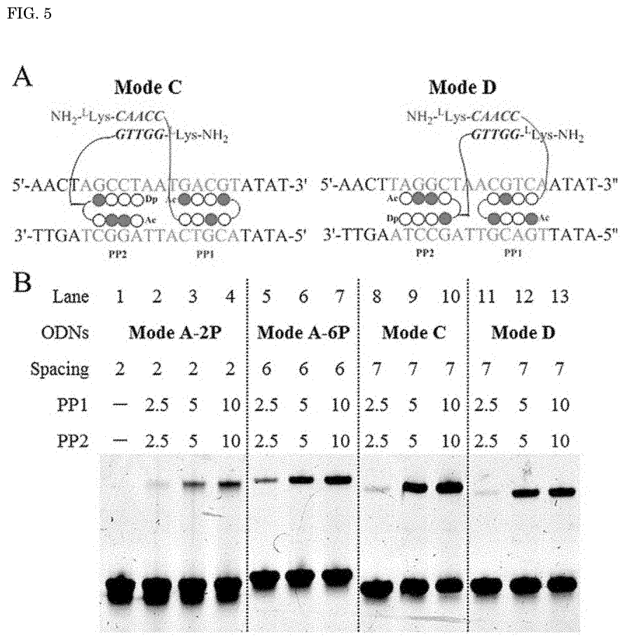

DNA-binding proteins can flexibly rearrange their binding orientations when coupled with partner TFs. See. Morgunova, J. Taipale, Curr. Opin. Struct. Biol. 2017, 47, 1-8. We have confirmed that PP1-PP2 possesses strong band-shift ability with ODNs of 0-13 nt spacings, which are long enough to accommodate the diverse binding modes of TF-DNA complexes. Here, we investigated PP1-PP2 complexed with ODNs in two additional binding modes, Modes C and D, to analyze the effects of orientation of PIP-binding sites on cooperative binding (FIG. 5, part A).

The results shown in FIG. 5, part B suggested that the order of binding affinity of the complexes is Mode A-2P<Mode A-6P<Mode D<Mode C. Because γPNA modules bind head-to-tail, the large size of the dimerization domain imposes unfavorable steric hindrance for Mode A-2P (with a spacing of 2 nt). Such steric hindrance is relieved when the distance increases to six or seven base pairs. Furthermore, Modes C and D both showed higher binding affinity than Mode A-6P, implying that a compact binding mode helped to stabilize the complexes. A slightly higher binding affinity of Mode D (5.0 μM, 29.1%) compared with Mode E (5.0 μM, 15.6%) might be explained by the difference of DNA sequence orientation. See A. Jolma, Y. Yin, K. R. Nitta, K. Dave, A. Popov, M. Taipale, M. Enge, T. Kivioj a, E. Morgunova, J. Taipale, Nature 2015, 527, 384-388.

### Energetics of Cooperative Binding

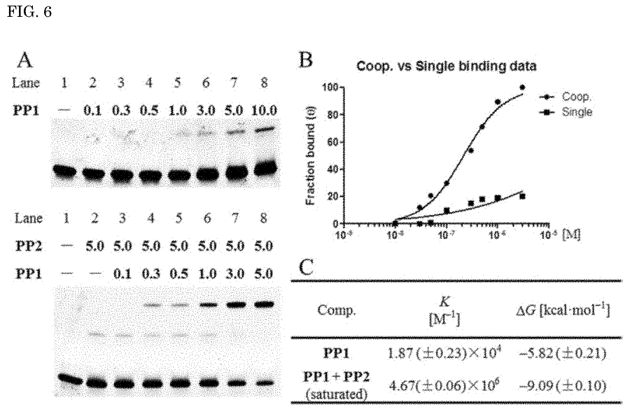

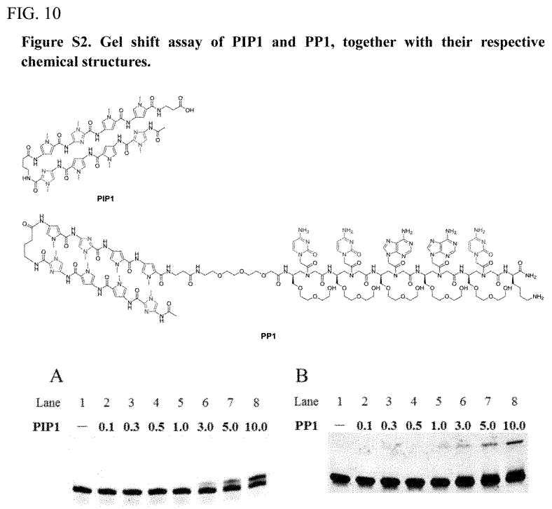

Quantitative EMSAs were performed to analyze the magnitude of cooperativity. See M. D. Distefano, P. B. Dervan, Proc. Natl. Acad. Sci. USA 1993, 90, 1179-1183; and R. Moretti, L. J. Donato, M. L. Brezinski, R. L. Stafford, H. Hoff, J. S. Thorson, P. B. Dervan, A. Z. Ansari, ACS Chem. Biol. 2008, 3, 220-229. The experimental design involved measuring the equilibrium constants for binding of PP1 to Mode C in the presence and absence of PP2. EMSA results confirmed that the conjugation of γPNA sequence moderately impairs PIPs binding affinity (FIG. 10). Incubation of ModeC with PP1 alone resulted in a very weak band-shift (FIG. 6, part A and FIG. 10).

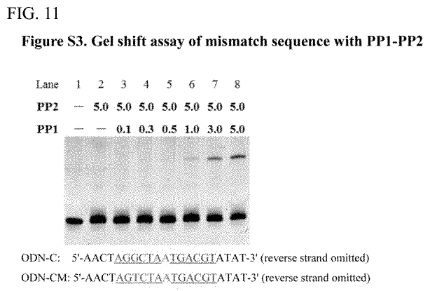

The increase in band-shift at low concentrations of PP1 alone and in the presence of 5.0 μM PP2 illustrates the cooperative effect. Compared with weak monomeric binding, γPNA dimerization domains facilitate dimeric binding to their respective biding sites. Fitting a Langmuir binding isotherm yielded the binding isotherms and equilibrium association constants of 1.87×10⁴M⁻¹ (Kᵢ) for PP1 binding alone and 4.67×10⁶M⁻¹ (K_{1,2}) for PP1 in the presence of 5.0 μM PP2 (FIG. 6, part B). Based on the free-energy-of-binding equation, we can calculate that the AG for PP1 in the presence and absence of PP2 is −9.09 and −5.82 kcal mol⁻¹, respectively. From this, we can estimate that the minimum free energy of interaction (ΔG_{1,2}−ΔG₁) is −3.27 kcal mol⁻¹ (FIG. 6, part C). Therefore, for this system, the presence of partner PP2 enhances the binding affinity of PP1 by a factor of more than 200. Pip-NaCo also showed high sequence selectivity in the assay with 1-bp mismatch DNA sequence (FIG. 11).

Even though Pip-NaCo show reasonable decreases of binding affinity by mono- or combinatory treatment compared with Pip-HoGu, Pip-NaCo revealed significant improvement on cooperation binding energy (from −2.32 to −3.27 kcal mol⁻¹) and further experiments demonstrated that cooperation strength can be regulated reasonably and flexible on the γPNA modules (see below).

### The Effect of PNA Length on Cooperative Binding

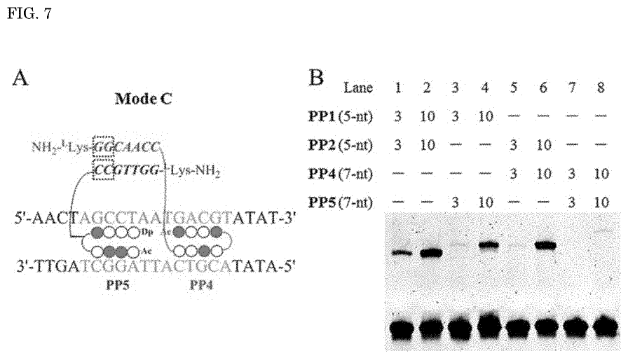

An important feature of the γPNA-based cooperative system is that the parallel γPNA dimerization domain can be tuned to regulate stabilization through alteration of the length and match/mismatch of PNA sequence. Here, we investigated the influence of PNA length on the cooperation of the Pip-NaCo assembly where the γPNA duplex is parallel to dsDNA. The 5 nt γPNA sequences in PP1 and PP2 were elongated to 7 nt to generate PP4 and PP5, respectively (FIG. 7, part A). After solid phase synthesis, 5 nt and 7 nt conjugates were evaluated using dimers of either the same γPNA length (5 nt:5 nt or 7 nt:7 nt) or mixed lengths (5 nt:7 nt).

The data showed the following order of binding affinity to Mode C: PP1-PP2>PP2-PP4>PP1-PP5>PP4-PP5, suggesting that the 7 nt γPNA conjugate destabilizes the binding compared with that of 5 nt γPNA (FIG. 7, part B). These data suggested that γPNA length was an important factor in regulating the binding of the complexes, and that for binding Mode C, a short γPNA might be preferable given that 5 nt γPNA has shown sufficiently potent duplex binding ability while further increase of γPNA length led to weak improvement on cooperation but might significantly reduce PIP-DNA binding affinity.^{[13]} One point to emphasize here is that we surmised that the larger size of the parallel form of the γPNA dimerization domain might easily displace PIPs from the DNA minor groove. It might be interesting to explore in the future vertical γPNA binding modes in which γPNA duplex is perpendicular to dsDNA, which has the potential to form more stable γPNA-assisted complexes (unpublished work). See M. D. Distefano, P. B. Dervan, Proc. Natl. Acad. Sci. USA 1993, 90, 1179-1183.

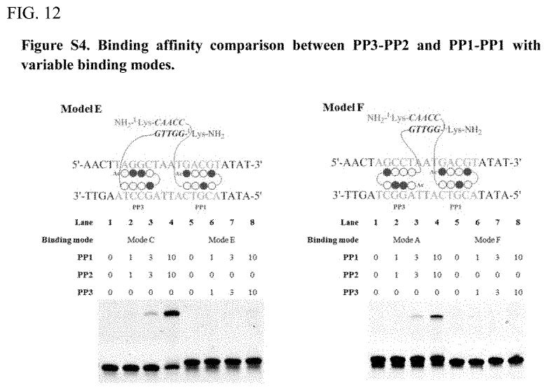

We also studied the influence of the linker conjugation site tethered with PIPs. In comparison with PP2, we designed PP3 in which the linker was conjugated at the tail of PIP2 rather than the gamma-turn (FIG. 12). The results demonstrated that this minor change in the conjugation site dramatically destabilized the interaction, suggesting that the conjugation site on the gamma-turn should be preserved.

### Competitive Assay

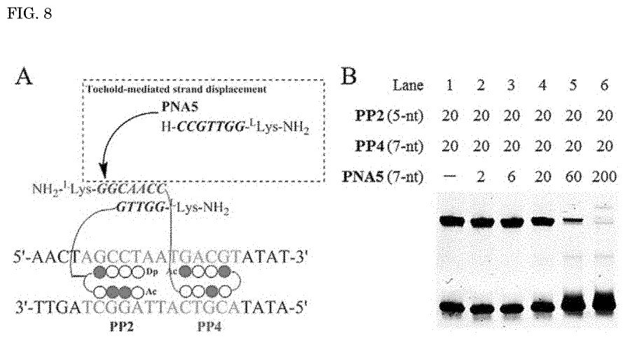

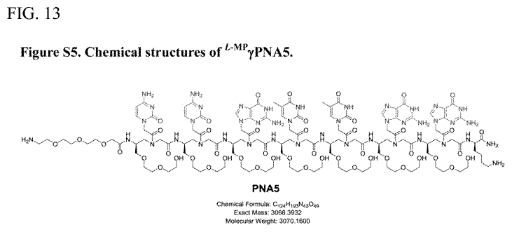

The feature of a toehold-mediated strand displacement assay has expanded the application of nucleic acid-based artificial systems. See D. Y. Zhang, G. Seelig, Nat. Chem. 2011, 3, 103-113. One advantage of the current artificial system derives from the reversibility of γPNA duplex formation depending on the composition of the external environment; for example, the presence of competitive γPNA strands. Here, we investigated the capabilities of the Pip-NaCo system in a competitive assay. Based on the theory of toehold-mediated strand displacement, a 7 nt PNA5 strand was introduced to displace PP4 binding (FIG. 8, part A and FIG. 13). PP2-PP4 complexes with a 5 nt:7 nt γPNA dimerization domain were stabilized with Mode C (lane 1, FIG. 8, part B). Concentration-dependent displacement by γPNA5 was observed during a short incubation, and at a threefold excess of γPNA5, >80% of PP4 was released from PP2-binding complexes (lane 5). This suggested that γPNA-based toehold-mediated strand displacement is of value for future applications in versatile, reversible artificial control systems.

### Conclusions

The important features of the artificial system Pip-NaCo characterized here are that both recognition domain PIPs and cooperative dimerization domain PNAs are modular, suggesting that they have controllable cooperative energetics. Through changing the linker conjugation site, binding mode, and sequence of PIPs and γPNAs, orientations of binding sites and cooperative-interaction energies can be tuned independently and reasonably. Moreover, the orthogonal properties of LH γPNA have the overwhelming advantage of eliminating the confusion generated by excess endogenous nucleic acids while maintaining its higher dimerization ability with its sequence-specific partner. Most significantly, Pip-NaCo has outstanding cooperative interaction ability compared with naturally occurring transcription factor pairs, and it can cover variable orientations of binding sites. The current Pip-NaCo platform also has the potential for precisely manipulating biological processes.

Experimental Section

Full experimental details are provided in the Supporting Information.

The supporting information is described as follows:

### Materials and Methods

### General

The reagents for polyamide syntheses such as Fmoc-Py-OH, Fmoc-Im-OH, Fmoc-Py-Im-OH, and Im-CC13, solid supports (Fmoc-Py-oxime resin and Fmoc-β Ala-Wang resin), O-(1H-6-chlorobenzotriazol-1-yl)-1,1,3,3-tetramethyluronium hexafluorophosphate (HCTU) and benzotriazol-1-yloxytripyrrolidinophosphonium hexafluorophosphate (PyBOP) were from HiPep Laboratories (Kyoto, Japan). Trifluoroacetic acid (TFA), 3,3′-diamino-N-methyldipropylamine, N,N-diisopropylethylamine (DIEA), dichloromethane (DCM), methanol, acetic acid (AcOH), 1-methyl-2-prrrolidone (NMP), and N,N-dimethylformamide (DMF) were obtained from Nacalai Tesque (Kyoto, Japan). Fmoc-D-Dab (Boc)-OH and Fmoc-NH-dPEG3-COOH were obtained from Iris Biotech GmbH (Marktredwitz, Deutschland). Polyamide-chain assembly was performed on an automated synthesizer, PSSM-8 (Shimadzu, Kyoto, Japan). HPLC grade acetonitrile (Nacalai tesque) was used for both analytical and preparative HPLC. Water was prepared by a Milli-Q apparatus (Millipore, Tokyo, Japan). All chemicals were used as received. Analyses by reversed-phase RP-HPLC were carried out online LCMS (Agilent 1100 ion-trap mass spectrometer, HCT ultra, Bruker Daltonics, Yokohama, Japan), with analytical RP-HPLC columns, UV spectra were measured on a NanoDrop 2000c (Thermo Fisher Scientific). ESI-MS and MALDI TOF-MS data for structural determination showed here are carried out in either Kyoto University or Carnegie Mellon University.

### Polyamide Fmoc Coupling Procedure

Polyamides were prepared using PSSM-8 peptide synthesizer (Shimadzu, Kyoto) with a computer-assisted operation system at 43 mg of Fmoc-Pyrrol-oxime resin and β Ala-Wang resin (ca. 0.42 mmol/g, 100˜200 mesh) by Fmoc solid-phase chemistry. See Z. Yu, J. Taniguchi, Y. Wei, G. N. Pandian, K. Hashiya, T. Bando, H. Sugiyama, Eur. J. Med. Chem. 2017, 138, 320-327; b) C. Guo, Y. Kawamoto, S. Asamitsu, Y. Sawatani, K. Hashiya, T. Bando, H. Sugiyama, Bioorg. Med. Chem. 2015, 23, 855-860. Reaction cycles were as follows: deblocking step for 4 min×2, 20% piperidine in DMF; coupling step for 60 min, corresponding carboxylic acids, HCTU (88 mg), diisopropylethylamine (DIEA) (36 μL), 1-methyl-2-pyrrolidone (NMP); washing step for 1 min×5, DMF. Each coupling reagents in steps were prepared in NMP solution of Fmoc-Py-COOH (77 mg), Fmoc-Im-COOH (77 mg), Fmoc-Py-Im-COOH (70 mg), Fmoc-β-COOH (66 mg), Fmoc-γ-COOH (69 mg) and Fmoc-mini PEG-COOH (69 mg). All other couplings were carried out with single-couple cycles with stirred by N2 gas bubbling. Typically, resin (40 mg) was swollen in 1 mL of NMP in a 2.5-mL plastic reaction vessel for 30 min. 2-mL plastic centrifuge tubes with loading Fmoc-monomers with HCTU in NMP 1 mL were placed in programmed position. All lines were washed with NMP after solution transfers. After the completion of the synthesis by the peptide synthesizer, the resin was washed with DMF (1 mL×2), methanol (1 mL×2), and dried in a desiccator at room temperature in vacuo.

### Resin Cleavage and Purification Procedure

The resulting polyamide-oxime resin was cleaved from the solid support with N,N-dimethyl-1,3-propyldiamine for 3 h at 45° C. Polyamide-β Ala-Wang resin was cleaved from the solid support with 95% TFA, 2.5% triisopropylsilane, and 2.5% water for 30 min at room temperature. Resin was filtered off, and the resulting liquor was treated with diethyl ether. The precipitated crude polyamide was washed three times with diethyl ether and analyzed by RP-HPLC. Crude polyamides were purified on a preparative column at 40° C. The purified peptides were assessed by the LC-MS system.

### PNA Monomer:

Detail synthetic route of each PNA monomer and PNA polymer can be found elsewhere of our previous work. See A. Manna, S. Rapireddy, G. Sureshkumar, D. H. Ly, Tetrahedron 2015, 71, 3507-3514.

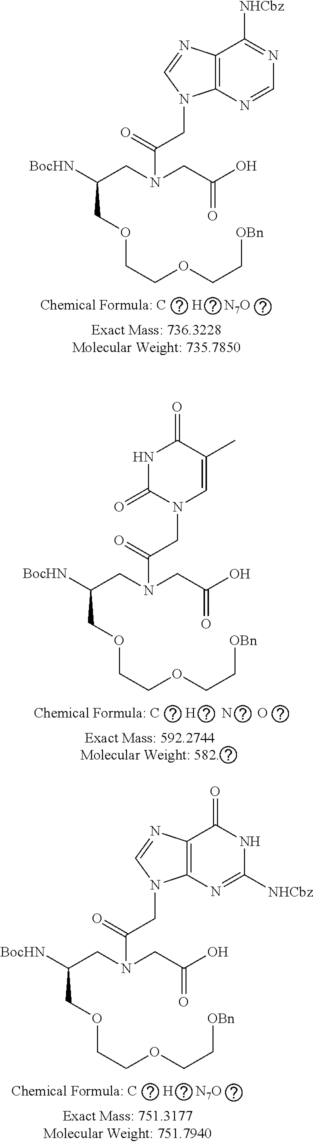 [SMILES: CC(C)(C)OC(=O)N[C@H](COCCOCCOCc1ccccc1)CN(CC(=O)O)C(=O)Cn1cnc2c(NC(=O)OCc3ccccc3)ncnc21 ; Cc1cn(CC(=O)N(CC(=O)O)C[C@@H](COCCOCCOCc2ccccc2)NC(=O)OC(C)(C)C)c(=O)[nH]c1=O ; CC(C)(C)OC(=O)N[C@H](COCCOCCOCc1ccccc1)CN(CC(=O)O)C(=O)Cn1cnc2c(=O)[nH]c(NC(=O)OCc3ccccc3)nc21]

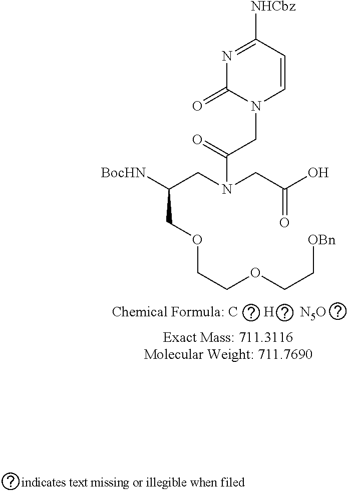 [SMILES: CC(C)(C)OC(=O)N[C@H](COCCOCCOCc1ccccc1)CN(CC(=O)O)C(=O)Cn1ccc(NC(=O)OCc2ccccc2)nc1=O]

Monomer pA: ESI-HRMS: m/z calcd for C36H45N7NaO10+[M+Na]+: 758.3126; found: 758.3114.
Monomer pT: ESI-HRMS: m/z calcd for C28H40N4NaO10+[M+Na]+: 615.2642; found: 615.2628.
Monomer pG: ESI-HRMS: m/z calcd for C36H45N7NaO11+[M+Na]+: 774.3075; found: 774.3060.
Monomer pC: ESI-HRMS: m/z calcd for C35H45N5NaO11+[M+Na]+: 734.3013; found: 734.3006.

### Synthesis of PIP1

Polyamide synthetic procedure has been described above. The resin cleavage and compound purification procedure have been described above. PIP1 was obtained as a white powder. Overall yield is 4.5%. MALDI-TOF MS: m/z calcd for C54H61N21NaO12+[M+Na]+: 1219.2068; found: 1218.608. HPLC: tR=16.675 min (0.1% TFA/MeCN, linear gradient 0-100%, 0-40 min). (Mass data was attached in the bottom)

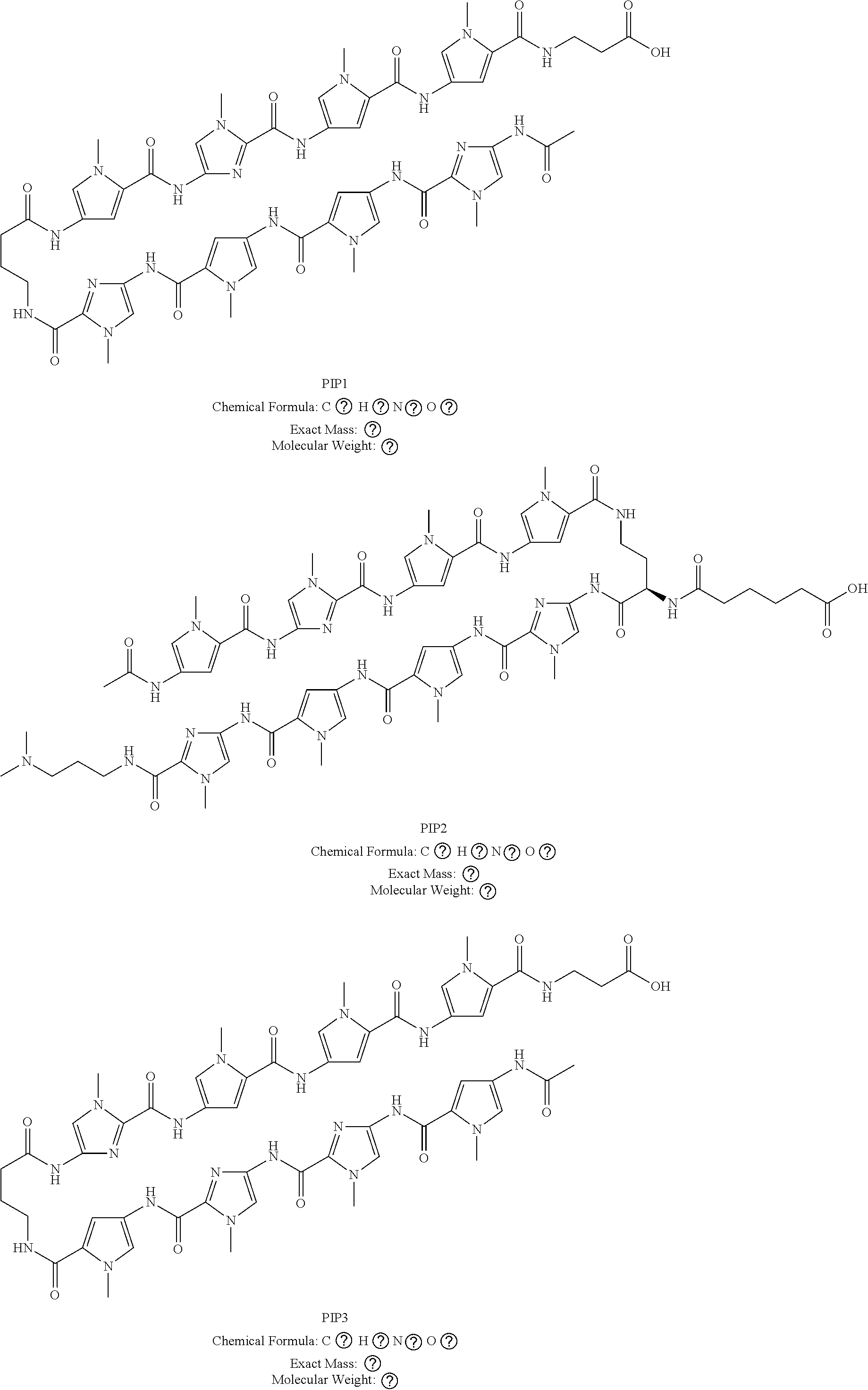

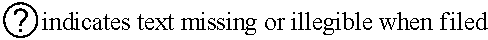

### Synthesis of PIP2

Polyamide synthetic procedure has been described above. The resin cleavage and compound purification procedure have been described above. PIP2 was obtained as a white powder. Overall yield is 13.5%. MALDI-TOF MS: m/z calcd for C62H78N23O13+[M+H]+: 1353.4540; found: 1351.968. HPLC: tR=9.875 min (0.1% TFA/MeCN, linear gradient 0-100%, 0-20 min). (Mass data was attached in the bottom)

### Synthesis of PIP3

Polyamide synthetic procedure has been described above. The resin cleavage and compound purification procedure have been described above. PIP3 was obtained as a white powder. Overall yield is 5.5%. MALDI-TOF MS: m/z calcd for C54H62N21O12+[M+H]+: 1197.2250; found: 1196.898. HPLC: tR=17.142 min (0.1% TFA/MeCN, linear gradient 0-100%, 0-40 min). (Mass data was attached in the bottom)

### Pip-PNA Synthesis[2]:

Synthetic Route of PP1 (Applied to PP1-PP5):

### Synthesis of PP1.

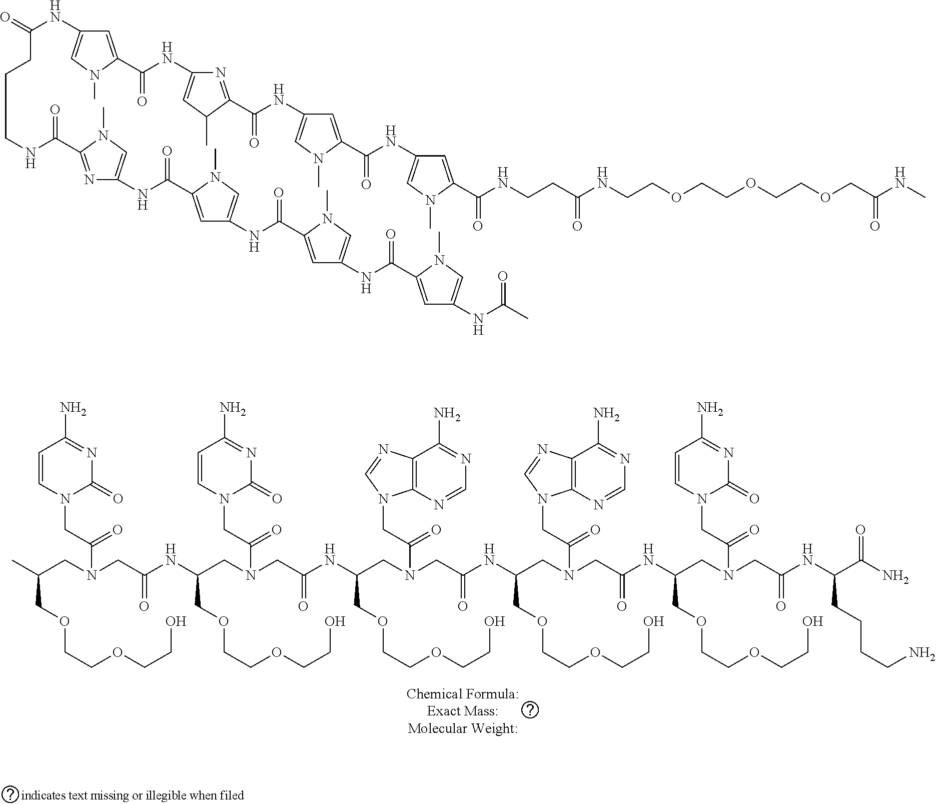

Synthetic route has been shown above. The resin cleavage and compound purification procedure have been described above. PP1 was obtained as a white powder. Yield is 35.1%. MALDI-TOF MS: m/z calcd for C145H205N54O44+[M+H]+: 3408.5690; found: 3405.703. HPLC: tR=26.283 min (0.1% TFA/MeCN, linear gradient 0-50%, 0-40 min). (Mass data was attached in the bottom)

### Synthesis of PP2

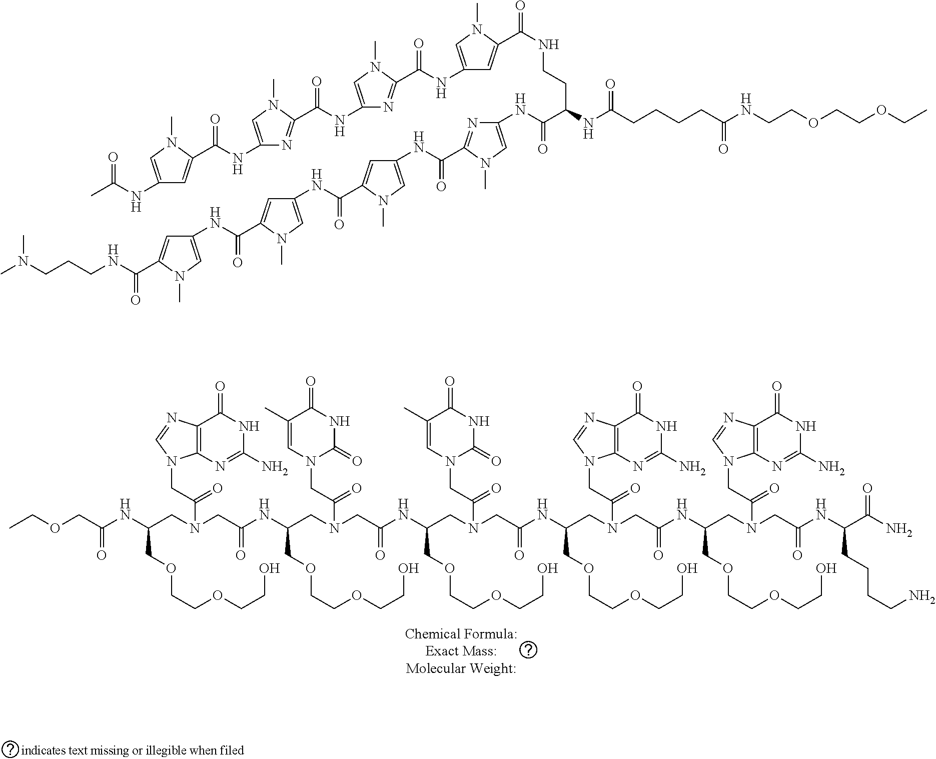

Synthetic route is similar with PP1. The resin cleavage and compound purification procedure have been described above. PP2 was obtained as a white powder. Yield is 27.1%. MALDI-TOF MS: m/z calcd for C156H223N56O49+[M+H]+: 3666.8430; found: 3664.700. HPLC: tR=26.990 min (0.1% TFA/MeCN, linear gradient 0-50%, 0-40 min). (Mass data was attached in the bottom)

### Synthesis of PP3

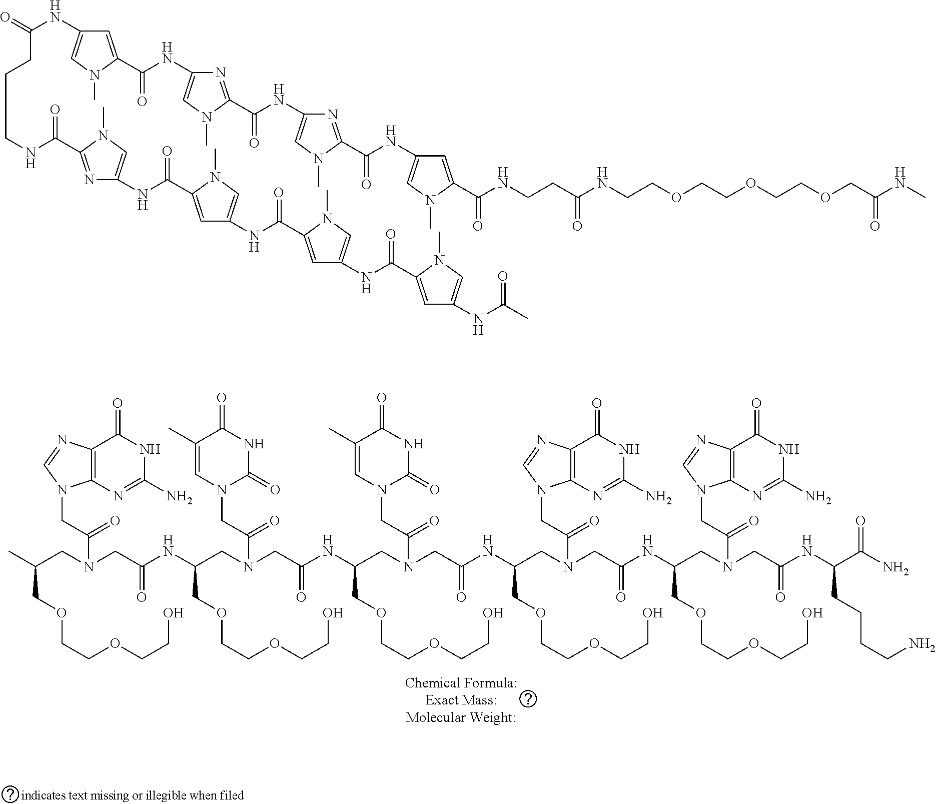

Synthetic route is similar with PP1. The resin cleavage and compound purification procedure have been described above. PP3 was obtained as a white powder. Yield is 25.9%. MALDI-TOF MS: m/z calcd for C148H207N54O48+[M+H]+: 3510.6140; found: 3509.891. HPLC: tR=27.200 min (0.1% TFA/MeCN, linear gradient 0-50%, 0-40 min). (Mass data was attached in the bottom)

### Synthesis of PP4

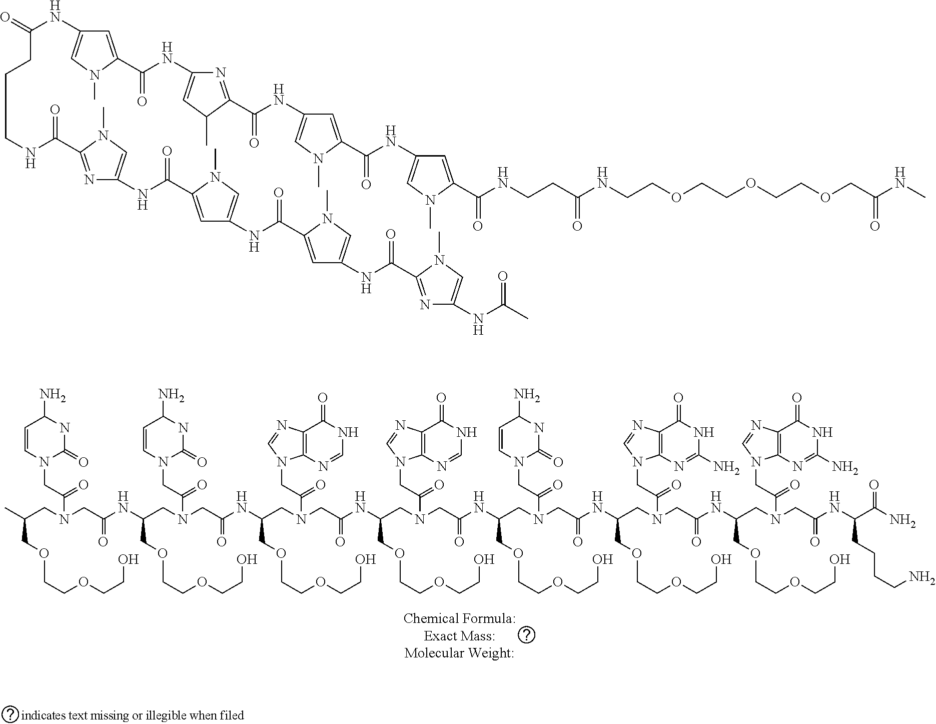

Synthetic route is similar with PP1. The resin cleavage and compound purification procedure have been described above. PP4 was obtained as a white powder. Yield is 36.5%. MALDI-TOF MS: m/z calcd for C177H260N68O68+[M+H]+: 4227.3750; found: 4226.890. HPLC: tR=26.083 min (0.1% TFA/MeCN, linear gradient 0-50%, 0-40 min). (Mass data was attached in the bottom)

### Synthesis of PP5

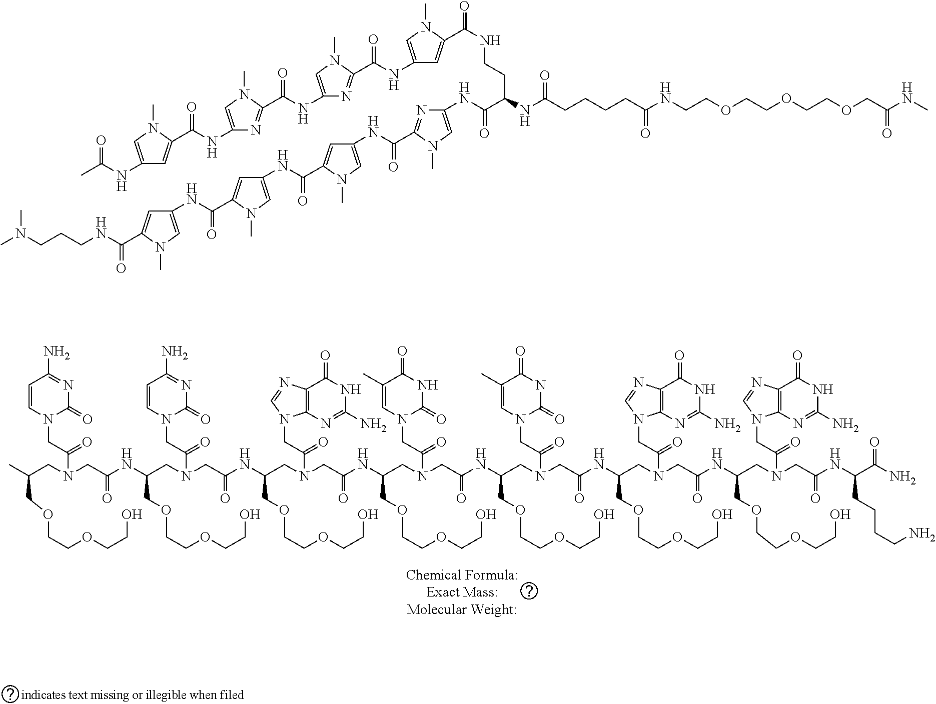

Synthetic route is similar with PP1. The resin cleavage and compound purification procedure have been described above. PP5 was obtained as a white powder. Yield is 28.1%. MALDI-TOF MS: m/z calcd for C186H268N66O61+[M+H]+: 4405.59900; found: 4405.149. HPLC: tR=26.242 min % TFA/MeCN, linear gradient 0-50%, 0-40 min). (Mass data was attached in the bottom)

### Compound Solution Preparation

Compounds were firstly dissolved in DMSO as the stock solution. PIPs and PIP-PNA conjugates concentrations were calculated with a Nanodrop ND-1000 spectrophotometer (Thermo Fisher Scientific Inc.) using an extinction coefficient of 9900 M-1 cm-1 per one pyrrole or imidazole moiety at max near 310 nm[1a]. Concentrations of PNA oligomers were determined from the OD at 260 nm recorded at 90° C., using the following extinction coefficient: T=8600 M-1 cm-1, A=13,700 M-1 cm-1, C=6600 M-1 cm-1, and G=11,700 M-1 cm-1. See A. Manna, S. Rapireddy, G. Sureshkumar, D. H. Ly, Tetrahedron 2015, 71, 3507-3514.

### Circular Dichroism (CD) Experiment

All PIPs and Pip-PNA conjugated was quantified as previous established methods of PIPs. See J. W. Trauger, E. E. Baird, P. B. Dervan, Nature 1996, 382, 559-561. The Pip-PNA samples (5 μM, 500 μL) for CD titration were prepared in 10 mM sodium phosphate, 0.1 mM EDTA, 100 mM NaCl, pH 7.2. Aliquots of master solution of compounds (1 mM in DMSO) were added continuously and incubated at least 3 min to reach the equilibrium. CD spectra were recorded at 22° C. over the range of 230-350 nm using JASCO J-805LST spectrometer in a 1-cm path length quartz cuvette.

### Electrophoretic Mobility Shift Assay (EMSA)

Preparation Loading Mixture.

See B. Heddi, V. V. Cheong, H. Martadinata, A. T. Phan, Proc. Natl. Acad. Sci. U.S.A. 2015, 112, 9608-9613. The sequences of the DNAs used were purchased from Sigma-Aldrich. The analysis buffer is as follows: the aqueous solution of 10 mM sodium phosphate, 100 mM NaCl, pH 7.2 containing 0.25% v/v DMSO. The final concentrations of polyamides and dsDNA were clarified in the manuscript. Mixtures were placed at room temperature for 2 h before gel loading. Gel Loading Dye was Purple 6X, no SDS (B70255, New England Bio lab).

Preparation of Gels.

In a clean glass beaker the following reagents were mixture in the given order (10 ml system, reagent volume doubled for 20 ml system). 5.25 mL MiliQ, 1 mL 10×TBE, and 3.75 mL of 40% Acrylamide/Bis Solution (29:1), followed by gas-removing to ensure the removal of all air bubbles. Then 90 μL APS (10% w/w in MiliQ) and 100 μL TEMED (10% v/v in MiliQ) were then added to the mixture and mixed properly before pouring it gently along parallel glass plates. Sufficient time was given for polymerization (20 min).

Electrophoresis.

A pre-run of the gels was performed prior to loading. Care was taken to see that the gel were properly immersed in 1×Tris-Borate-EDTA buffer (TBE buffer) and the loading wells were free from any air bubbles. The wells were washed after the pre-run. Instrument settings: 120 V for 30 min at 4° C. 4 μL of the loading mixture was then loaded onto the wells. Pre-run again at 120 V for 30 minutes at 4° C. Then gel running as the instrument settings: 180 V for 160 min at 4° C.

Analysis of Gels.

The bands were stained with SYBR gold (10000× concentration in DMSO, from Thermofisher) and quantified with a FujiFilm FLA-3000G fluorescent imaging analyzer. FAM labeled forward strand (5′-FAM-AACTAGCCTAATGACGTATAT-3′) (SEQ ID NO:1) was used for quantitative assay directly with a FujiFilm FLA-3000G fluorescent imaging analyzer without SYBR gold staining.

[symbol]

### Quantitative Determination of Minimum Cooperative Binding Energy

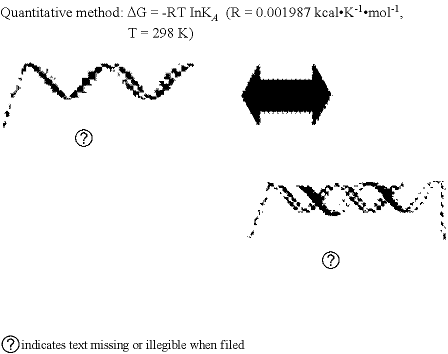

Quantitative EMSAs (FAM-labeled ODN) were performed to analyze the magnitude of cooperativity. See R. Moretti, L. J. Donato, M. L. Brezinski, R. L. Stafford, H. Hoff, J. S. Thorson, P. B. Dervan, A. Z. Ansari, ACS. Chem. Biol. 2008, 3, 220-229; and M. D. Distefano, P. B. Dervan, Proc. Natl. Acad. Sci. U.S.A. 1993, 90, 1179-1183. The experimental design involved measuring the equilibrium constants for binding of PP1 to Mode C in the presence and absence of PP2. Fitting a Langmuir binding isotherm yielded the binding isotherms and equilibrium association constants of K1 for PP1 binding alone and K1,2 for PP1 in the presence of PP2. Based on the free-energy-of-binding equation, we can calculate that the ΔG2 and ΔG2-1 for PP1 in the presence and absence of PP2, respectively. From this, we can estimate that the minimum free energy of interaction (ΔG1,2−ΔG1). GraphPad Prism 5 were used for curve fitting lead to the calculation of equilibrium association constant. Gas constant (R) is 0.001987 kcal·K−1·mol−1 and T=298 K.

### Statistical Analysis

Results for continuous variables were presented as the mean±standard error. Two-group differences in continuous variables were assessed by the unpaired T-test. Statistical analysis was performed by comparing treated samples with untreated controls. The statistical analyses were performed using GraphPad Prism 5.

### Supporting Table

TABLE S1
Detailed information of the relationship among gap distance, moiety
distance, and propeller angle.

| DNA mode | Mode A | | | | | | | | | Mode B | | | | | | | | |
|---|---|---|---|---|---|---|---|---|---|---|---|---|---|---|---|---|---|---|
| ODNs | 1P | 0P | 1P | 2P | 3P | 4P | 5P | 6P | 8P | 1N | 0N | 1N | 2N | 3N | 4N | 5N | 6N | 8N |
| Gap distance | −1 | 0 | 1 | 2 | 3 | 4 | 5 | 6 | 8 | 11 | 12 | 13 | 14 | 15 | 16 | 17 | 18 | 20 |
| Spacing | 1 | 2 | 3 | 4 | 5 | 6 | 7 | 8 | 10 | 1 | 2 | 3 | 4 | 5 | 6 | 7 | 8 | 10 |
| Propeller angle | — | 36 | 72 | 108 | 144 | 180 | 216 | 252 | 288 | 36 | 72 | 108 | 144 | 180 | 216 | 252 | 288 | 360 |

1. A conjugate of a pair of DNA binders with a nucleic acid-based cooperation domain.

2. The conjugate according to claim 1, wherein the DNA binders are pyrrole-imidazole polyamides.

3. The conjugate according to claim 1, wherein the nucleic acid-based cooperation domain comprises left-handed (LH) gamma-PNA.

4. The conjugate according to claim 2, wherein the nucleic acid-based cooperation domain comprises left-handed (LH) gamma-PNA.


SEQUENCE LISTING (23 sequences)

SEQ ID NO: 1 - DNA, 21 bp, Artificial
  ODN
aactagcctaatgacgtatat

SEQ ID NO: 2 - DNA, 21 bp, Artificial
  ODN
aactaggctaatgacgtatat

SEQ ID NO: 3 - DNA, 21 bp, Articial
  ODN
aactagtctaatgacgtatat

SEQ ID NO: 4 - DNA, 22 bp, Artificial
  ODN
aacttaggctaatgacgtatat

SEQ ID NO: 5 - DNA, 22 bp, Artificial
  ODN
aaacttaggctgacgtatatat

SEQ ID NO: 6 - DNA, 22 bp, Artificial
  ODN
aaacttaggcttgacgtatata

SEQ ID NO: 7 - DNA, 22 bp, Artificial
  ODN
aacttaggctatgacgtatata

SEQ ID NO: 8 - DNA, 22 bp, Artificial
  ODN
aacttaggctaatgacgtatat

SEQ ID NO: 9 - DNA, 23 bp, Artificial
  ODN
aacttaggctaaatgacgtatat

SEQ ID NO: 10 - DNA, 24 bp, Artificial
  ODN
aacttaggctattatgacgtatat

SEQ ID NO: 11 - DNA, 24 bp, Artificial
  ODN
aattaggctattaatgacgtatat

SEQ ID NO: 12 - DNA, 24 bp, Artificial
  ODN
aattaggctaattaatgacgtata

SEQ ID NO: 13 - DNA, 26 bp, Artificial
  ODN
aattaggctggaattcctgacgtata

SEQ ID NO: 14 - DNA, 22 bp, Artificial
  ODN
aaacttgacgtaggctatatat

SEQ ID NO: 15 - DNA, 22 bp, Artificial
  ODN
aaacttgacgttaggctatata

SEQ ID NO: 16 - DNA, 22 bp, Artificial
  ODN
aacttgacgtataggctatata

SEQ ID NO: 17 - DNA, 22 bp, Artificial
  ODN
aacttgacgtaataggctatat

SEQ ID NO: 18 - DNA, 23 bp, Artificial
  ODN
aacttgacgtaaataggctatat

SEQ ID NO: 19 - DNA, 24 bp, Artificial
  ODN
aacttgacgtattataggctatat

SEQ ID NO: 20 - DNA, 24 bp, Artificial
  ODN
aattgacgtattaataggctatat

SEQ ID NO: 21 - DNA, 24 bp, Artificial
  ODN
aattgacgtaattaataggctata

SEQ ID NO: 22 - DNA, 26 bp, Artificial
  ODN
aattgacgtggaattcctaggctata

SEQ ID NO: 23 - DNA, 21 bp, Artificial
  ODN
aacttaggctaacgtcaatat